(19) United States
(12) Patent Application Publication
(10) Pub. No.: US 2020/0179622 A1
(43) Pub. Date: Jun. 11, 2020

(54) ATOMIZER

(71) Applicant: Airplove (Xiamen) Electronic Co., Ltd., Xiamen (CN)

(72) Inventors: Jueyuan CHEN, Xiamen (CN); Yang ZHOU, Xiamen (CN); Yunlong GAO, Xiamen (CN)

(21) Appl. No.: 16/418,375

(22) Filed: May 21, 2019

(30) Foreign Application Priority Data
Dec. 10, 2018 (CN) 201822062030.1
Mar. 30, 2019 (CN) 201920426543.8

(51) Int. Cl. A61M 11/04; A61M 16/06

(52) U.S. Cl. CPC A61M 11/042; A61M 2205/3372; A61M 2205/0238; A61M 16/06

(57) ABSTRACT

An atomizer includes a shell, an electronic system and a heating body. The heating body includes an atomization piece of a flat structure and a heating film attached to the bottom surface of the atomization piece. The shell has a receiving chamber. The atomization piece is matched with the bottom of the receiving chamber in shape and is laid at the bottom of the receiving chamber in a sealing manner. The bottom of the receiving chamber is hollowed out, exposing the heating film. The electronic system in the shell is electrically connected with the heating film. A heating body of a columnar structure of existing atomizers is replaced with the atomization piece, and the atomization piece is matched with the bottom of the receiving chamber in shape and is laid at the bottom, so that a surface heating structure easy to clean is formed, liquid is uniformly heated and atomized.

### BACKGROUND OF THE INVENTION

### Technical Field

The invention relates to an atomizer, in particular to an atomizer which is simple in structure and easy to clean.

### 2. Description of Related Art

Atomizers are used to generate aerosols by atomizing water, saline water, and liquid medicine for treating bronchogenic diseases or other diseases. Common atomizers typically include a receiving chamber used for receiving liquids and a heating body arranged in the receiving chamber, wherein the heating body is generally of a columnar structure and protrudes out of the bottom of the receiving chamber. The atomizers of such structure have the disadvantage that a small gap between the heating body of the columnar structure and the wall of the receiving chamber is inconvenient to clean, so that bacteria easily breed in this gap and will do harm to the health of users in long-time use.

### BRIEF SUMMARY OF THE INVENTION

The objective of the invention is to provide an atomizer which is simple in structure and easy to clean.

The technical solution adopted by the invention to fulfill the above objective is as follows:

An atomizer comprises a shell, an electronic system and a heating body. The heating body comprises an atomization piece of a flat structure and a heating film attached to the bottom surface of the atomization piece. The shell is provided with a receiving chamber. The atomization piece is matched with the bottom of the receiving chamber in shape and is laid at the bottom of the receiving chamber in a sealing manner. The bottom of the receiving chamber is hollowed out to form a through hole. The heating film is exposed out of the through hole. The electronic system is arranged in the shell and is electrically connected with the heating film.

Furthermore, a metal heat-conduction layer is laid on the inner wall of the receiving chamber and the outer edge of the atomization piece in a sealing manner.

Furthermore, the metal heat-conduction layer is a copper foil layer.

Furthermore, a heat-conduction cup is arranged in the receiving chamber of the shell, and the atomization piece is attached to the bottom surface of the heat-conduction cup.

Furthermore, the outer wall of the heat-conduction cup is matched with the inner wall of the receiving chamber.

Furthermore, the heat-conduction cup is detachably installed in the receiving chamber of the shell.

Furthermore, the heat-conduction cup is provided with first installation structures, the receiving chamber of the shell is provided with second installation structures, and the heat-conduction cup is installed in the receiving chamber through the cooperation between the first installation structures of the heat-conduction cup and the second installation structures of the receiving chamber.

Furthermore, the first installation structures are second connectors arranged on the outer wall of the heat-conduction cup, and the second installation structures are L-shaped clamping grooves formed in the inner wall of the receiving chamber; each L-shaped clamping groove comprises a second vertical groove penetrating through the upper end face of the atomizer base and a second horizontal groove connected with the second vertical groove; and the second connectors are inserted via the second vertical grooves to be clamped into the second horizontal grooves.

Furthermore, limiting slots matched with the second connectors are formed in the tail ends of the second horizontal grooves, limiting ribs protruding out of the bottoms of the second horizontal grooves are arranged at middle positions of the second horizontal grooves, and under the effect of an external force, the second connectors surmount the limiting ribs to be clamped in the limiting slots of the second horizontal grooves.

Furthermore, openings, penetrating through the upper end face of atomizer base, of the second vertical grooves are open.

Furthermore, the heat-conduction cup is made from copper foil.

Furthermore, the atomization piece and receiving chamber are connected through integrated filling formation.

Furthermore, the atomization piece is a ceramic atomization piece.

Furthermore, the shell comprises a base, an annular connecting base and an atomizer base, wherein the base is provided with a receiving cavity, an opening penetrating through the receiving cavity is formed in an upper portion of the base, the receiving chamber is formed in the atomizer base which is fixedly connected to the opening of the base through the annular connecting base, and the electronic system is arranged in the receiving cavity of the base.

Furthermore, the base is provided with heat dissipation slots.

Furthermore, the number of the heat dissipation slots is more than one, and the heat dissipation slots are annularly arrayed on the base.

Furthermore, barrier parts are integrally formed at positions, directly facing the heat dissipation slots, of the base, and each barrier part is provided with a lateral opening.

Furthermore, each barrier part is further provided with an arc-shaped inner wall located between the corresponding heat dissipation slot and the lateral opening of the barrier part.

Furthermore, the base is composed of a lower cover and an upper cover, the receiving cavity is defined by the lower cover and the upper cover jointly, the opening is formed in the upper cover, and the heat dissipation slots are formed in the lower cover.

Furthermore, clamping grooves are concavely formed in the outer wall of the atomizer base.

Furthermore, a switch button is arranged on the shell and is connected with the electronic system.

By adoption of the technical solution, the invention has the following beneficial effects:

A heating body of a columnar structure of existing atomizers is replaced with the atomization piece of the flat structure in this solution, and the atomization piece is matched with the bottom of the receiving chamber in shape and is laid at the bottom of the receiving chamber in the sealing manner, so that a surface heating structure is formed, the heating area is enlarged, and liquid is uniformly heated and atomized, and a better atomization effect is realized. Compared with the heating body of the convex columnar structure, the atomization piece of the flat structure has a larger space and is easier to clean. The through hole is formed in the bottom of the receiving chamber, and the heating film is connected with the atomization piece and the electronic system via the through hole, so that the structure is simple, connection is convenient, and the design is reasonable.

### BRIEF DESCRIPTION OF THE SEVERAL VIEWS OF THE DRAWINGS

FIG. 1 is a perspective view of an atomizer in embodiment 1;

FIG. 2 is an exploded view of the atomizer in embodiment 1;

FIG. 3 is a sectional view of the atomizer in embodiment 1;

FIG. 4 is a structural view of an atomizer base of the atomizer in embodiment 1;

FIG. 5 is a structural view of a lower cover of the atomizer in embodiment 1;

FIG. 6 is a perspective view of an atomizer in embodiment 2;

FIG. 7 is a sectional view of the atomizer in embodiment 2;

FIG. 8 is an exploded view of the atomizer in embodiment 2;

FIG. 9 is a partial exploded view of the atomizer in embodiment 2;

FIG. 10 is a structural view of a heat-conduction cup hidden in the atomizer in embodiment 2;

FIG. 11 is a structural view of the heat-conduction cup of the atomizer in embodiment 2;

FIG. 12 is a structural view of a lower cover of the atomizer in embodiment 2;

FIG. 13 is a structural view of an atomizer base of the atomizer in embodiment 2; and

FIG. 14 is a structural view of one specific application of an atomizer in embodiment 3.

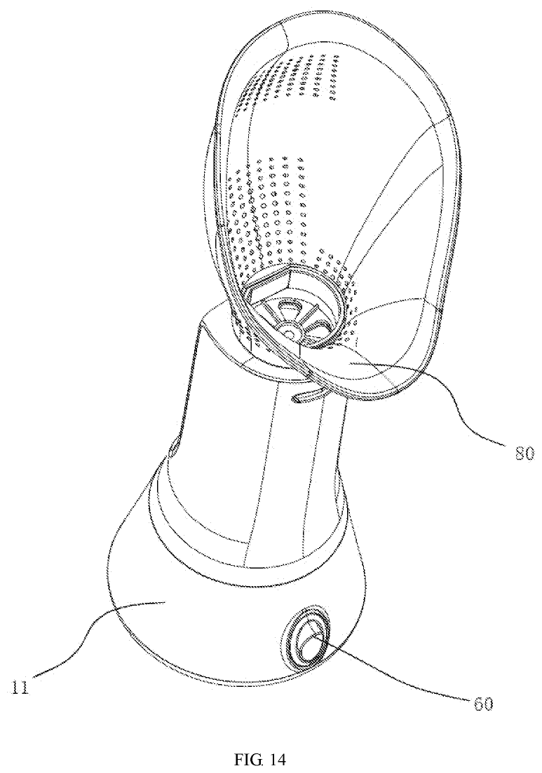

### DETAILED DESCRIPTION OF THE INVENTION

The accompanying drawings are provided for a further explanation of embodiments of the invention. These drawings are part of the contents of this disclosure and are mainly used to illustrate the embodiments and to explain the operation principle of these embodiments in cooperation with relevant contents in the description. Those ordinarily skilled in this field can appreciate other possible implementations and advantages of the invention by referring to these contents. Components in the drawings are not drawn to scale, and similar reference signs usually represent similar components.

The invention is further explained below with reference to the accompanying drawings and specific embodiments.

### Embodiment 1

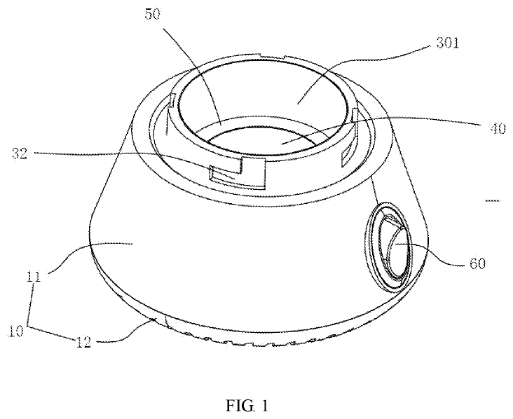

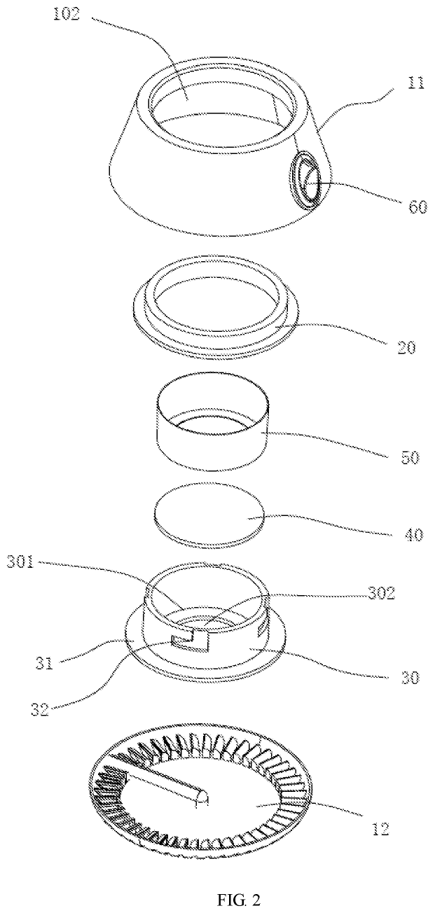

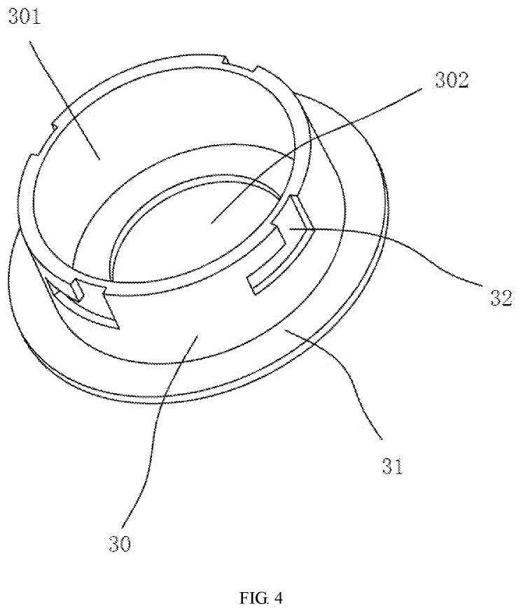

Referring to FIGS. 1-5, the invention provides an atomizer. The atomizer comprises a shell, an electronic system (not shown) and a heating body. The heating body comprises an atomization piece 40 of a flat structure and a heating film (not shown) attached to the bottom surface of the atomization piece 40. The shell is provided with a receiving chamber 301. The atomization piece 40 is matched with the bottom of the receiving chamber 301 in shape and is laid at the bottom of the receiving chamber 301 in a sealing manner. The bottom of the receiving chamber 301 is hollowed out to form a through hole 302. The heating film is exposed out of the through hole 302. The electronic system is arranged in the shell and is electrically connected with the heating film so as to control the heating film to generate heat which is in turn transferred to the atomization piece 40, and then liquid in the receiving chamber 301 is atomized by the atomization piece 40.

A heating body of a columnar structure of existing atomizers is replaced with the atomization piece 40 of the flat structure in this solution, and the atomization piece 40 is matched with the bottom of the receiving chamber 301 in shape and is laid at the bottom of the receiving chamber 301 in the sealing manner, so that a surface heating structure which is easy to clean is formed, liquid is uniformly heated and atomized, and a better atomization effect is realized.

Furthermore, a metal heat-conduction layer 50 is laid on the inner wall of the receiving chamber 301 and the outer edge of the atomization piece 40 in a sealing manner. Part of the heat from the atomization piece 40 can be evenly transmitted by the metal heat-conduction layer 50 to uniformly heat the to-be-atomized liquid in a large area, and thus, the liquid is atomized more easily and efficiently. Specifically, the metal heat-conduction layer 50 is a copper foil layer which is good in heat conductivity and stable in texture and structure. Definitely, the copper foil layer can be replaced with other metal materials in other embodiments.

Furthermore, the atomization piece 40 is a ceramic atomization piece which is good in heat conductivity, stable in structure, and long in service life.

Furthermore, the atomization piece 40 and the receiving chamber 301 are connected through an integrated filling formation process in the prior art, thereby being good in connection airtightness. Definitely, in other embodiments, the atomization piece 40 and the receiving chamber 301 can also be subsequently assembled with a sealant or through other sealing methods.

Furthermore, in this embodiment, the shell comprises a base 10, an annular connecting base 20 and an atomizer base 30, wherein the base 10 is provided with a receiving cavity 101, an opening 102 penetrating through the receiving cavity 101 is formed in an upper portion of the base 10, the receiving chamber 301 is formed on the atomizer base 30 which is fixedly connected to the opening 102 of the base 10 through the annular connecting base 20, and the electronic system is arranged in the receiving cavity 101 of the base 10.

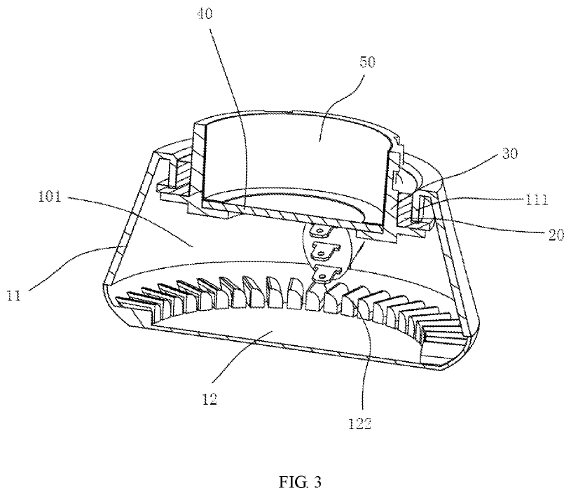

Particularly, the atomizer base 30 is of a cylindrical structure, and a step part 31 extending outwards is formed on the outer wall of the bottom end of the atomizer base 30. The section of the annular connecting base 20 is in an L shape, and as shown in FIG. 3, the inner wall and the bottom surface of the annular connecting part 20 are fixedly attached to the step part 31 of the atomizer base 30. A connecting part 111 extending downwards is arranged at the opening 102 of the base 10, and the outer wall of the annular connecting base 20 is fixedly attached to the connecting part 111 at the opening 101 of the base 10, so that the base 10, the annular connecting base 20, and the atomizer base 30 are fixedly connected. Definitely, in other embodiments, the base 10, the annular connecting base 20, and the atomizer base 30 of the shell can also be connected through other connecting structures.

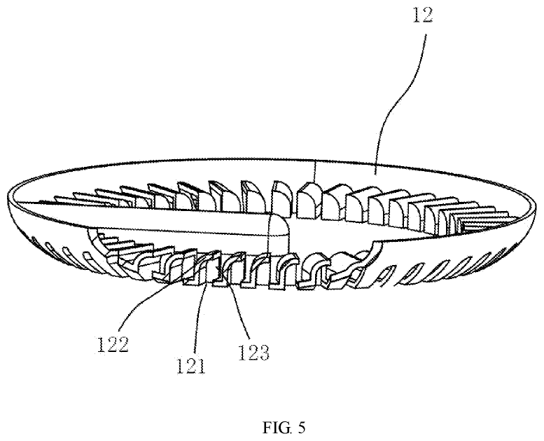

More particularly, the base 10 is composed of a lower cover 12 and an upper cover 11, and the opening 102 is formed in the upper cover 11. As shown in FIG. 5, the lower cover 12 is provided with heat dissipation slots 121 which are annularly arrayed, barrier parts 122 are integrally formed at positions, directly facing the heat dissipation slots 121, of the lower cover 12 of the base 10. Each barrier part 122 is provided with a lateral opening 123. The barrier parts 122 prevent the heat dissipation slots 121 from directly facing the receiving cavity 101, so that a good dust prevention effect is realized. When heat is dissipated, an external air flow enters the heat dissipation slots 121 and then flows into the receiving cavity 101 via the lateral openings 123, and an internal air flow enters the lateral openings 123 of the barrier parts 122 and then flows out via the heat dissipation slots 121.

Furthermore, in this embodiment, clamping grooves 32 are concavely formed in the outer wall of the atomizer base 30 and are used for fixedly clamping a hood or other connecting structures.

Furthermore, in this embodiment, a switch button 60 is arranged on the shell and is connected with the electronic system to control the operation of the atomizer.

Particularly, the electronic system and the heating film of the atomizer are well known in the prior art and are common structures of existing atomizers (such as electronic cigarettes and humidifiers), the structure of controlling the operation of the atomizer by the switch button connected with the electronic system is also well known in the prior art and has already been mastered and actually applied by those skilled in this field, and thus, specific constituent parts and connection relations of these structures will not be detailed anymore.

### Embodiment 2

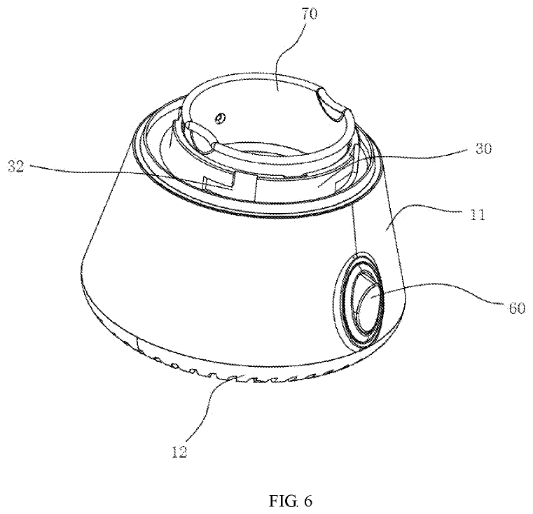

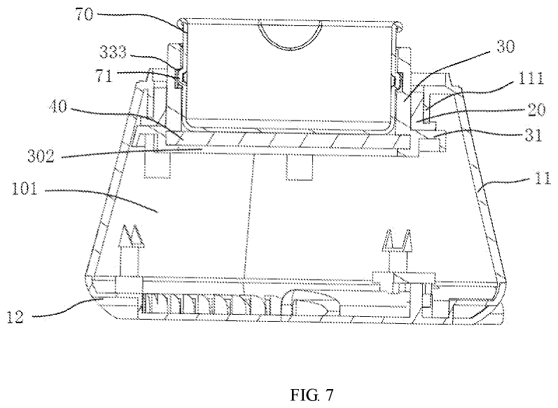

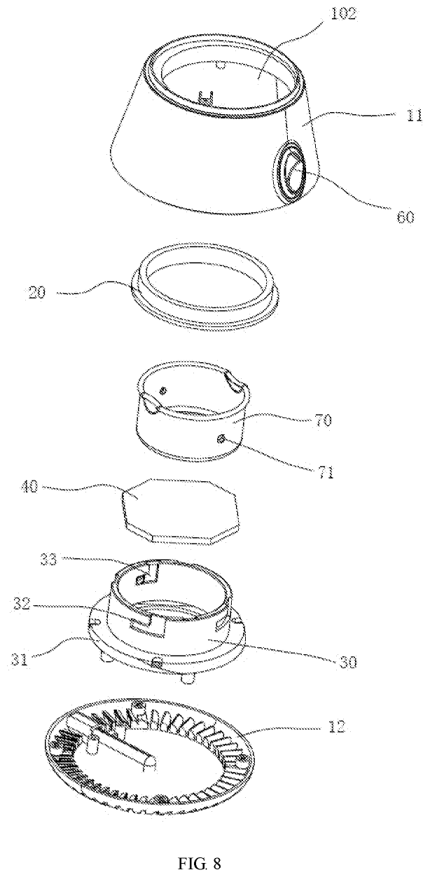

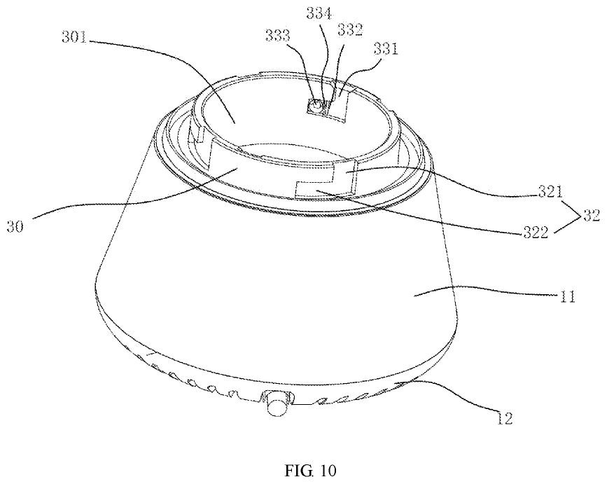

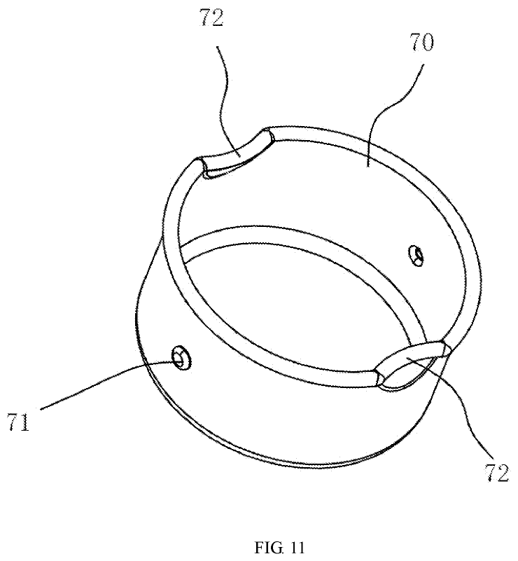

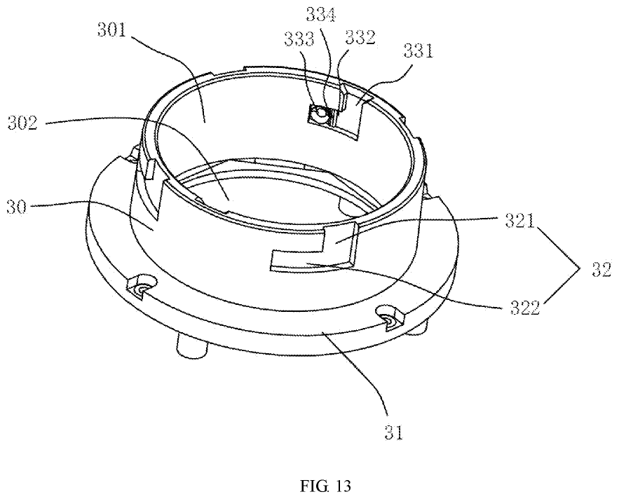

Referring to FIGS. 6-13, the invention provides an atomizer. The atomizer comprises a shell, an electronic system (not shown) and a heating body. The heating body comprises an atomization piece 40 of a flat structure and a heating film (not shown) attached to the bottom surface of the atomization piece 40. The shell is provided with a receiving chamber 30, and the atomization piece 40 is matched with the bottom of the receiving chamber 301 in shape and is laid at the bottom of the receiving chamber 301 in a sealing manner. The bottom of the receiving chamber 301 is hollowed out to form a through hole 302, and the heating film is exposed out of the through hole 302. The electronic system is arranged in the shell and is electrically connected with the heating film so as to control the heating film to generate heat which is in turn transferred to the atomization piece 40, and then liquid in the receiving chamber 301 is atomized by the atomization piece 40.

A heating body of a columnar structure of existing atomizers is replaced with the atomization piece 40 of the flat structure in this solution, and the atomization piece 40 is matched with the bottom of the receiving chamber 301 in shape and is laid at the bottom of the receiving chamber 301 in the sealing manner, so that a surface heating structure is formed, the heating area is enlarged, the liquid is uniformly heated and atomized, and a better atomization effect is realized. Compared with the heating body of the convex columnar structure, the atomization piece 40 of the flat structure has a larger space and is easier to clean. The through hole 302 is formed in the bottom of the receiving chamber 301, and the heating film is connected with the atomization piece 40 and the electronic system via the through hole 302, so that the structure is simple, connection is convenient, and the design is reasonable. This embodiment fulfills the same technical effects as embodiment 1.

Furthermore, in this embodiment, a heat-conduction cup 70 is arranged in the receiving chamber 301 of the shell, and the atomization piece 40 is attached to the bottom surface of the heat-conduction cup 70. Namely, the metal heat-conduction layer in embodiment 1 is replaced with the heat-conduction cup 70 used for containing to-be-atomized liquid, and the atomization piece 40 of the flat structure forms a surface heating structure, so that the heating area is enlarged; heat from the atomization piece is transferred onto the heat-conduction cup 70, then the to-be-atomized liquid is heated by the heat-conduction cup 70, and in this way, heat is evenly transferred, the liquid is uniformly heated and atomized, and a better atomization effect is realized; the heat-conduction cup 70 prevents the atomization piece 40 from directly making contact with liquid and can be directly cleaned, so that cleaning is easier; and the surface of the atomization piece 40 or the joints of the atomization piece 40 and other structures (such as the joint of the atomization piece and the metal heat-conduction layer in embodiment 1) are shielded against residues or other pollutants which are difficult to remove.

Furthermore, in this embodiment, the outer wall of the heat-conduction cup 70 is matched with the inner wall of the receiving chamber 301, so that the heat-conduction cup 70 and the receiving chamber 301 are adaptively connected and are more convenient to assemble and more stable in structure.

Furthermore, in this embodiment, the heat-conduction cup 70 is detachably installed in the receiving chamber 301 of the shell. As the heat-conduction cup 70 is detachably installed in the receiving chamber 301, the heat-conduction cup 70 can be independently disassembled to be cleaned more conveniently and thoroughly and can be directly assembled after being cleaned, so that normal usage is not affected. Or, a plurality of heat-conduction cups 70 are alternately used. When needing to be cleaned, the heat-conduction cup 70 being used is disassembled and is replaced with another clean heat-conduction cup 70, so that continuous usage is ensured.

Furthermore, in this embodiment, the heat-conduction cup 70 is provided with first installation structures, the receiving chamber 301 of the shell is provided with second installation structures, and the heat-conduction cup 70 is installed in the receiving chamber 301 through the cooperation between the first installation structures of the heat-conduction cup 70 and the second installation structure of the receiving chamber 301. The heat-conduction cup 70 is fixedly arranged in the receiving chamber 301 to stably make contact with the atomization piece 40, so that heat is stably transferred.

Particularly, the first installation structures are second connectors 71 arranged on the outer wall of the heat-conduction cup 70, and the second installation structures are L-shaped clamping grooves 33 formed in the inner wall of the receiving chamber 301. Each L-shaped clamping groove 33 comprises a second vertical groove 331 penetrating through the upper end face of the atomizer base 30 and a second horizontal groove 332 connected with the second vertical groove 331. When the heat-conduction cup 70 is placed in the receiving chamber 301, the second connectors 71 are inserted via openings of the second vertical grooves 331 to reach the bottom and are then rotated to be clamped in the second horizontal grooves 332, and then the heat-conduction cup 70 is clamped and fixed in the receiving chamber 301. This structure facilitates connection and is stable. In other embodiments, the heat-conduction cup 70 and the receiving chamber 301 can be connected in other ways such as buckling and screwing.

Furthermore, in this embodiment, the openings, penetrating through the upper end face the atomizer base 30, of the second vertical grooves 331 are open to fulfill a guide function, so that assembly and connection are more convenient and accurate.

Furthermore, in this embodiment, limiting slots 333 matched with the second connectors 71 are formed in the tail ends of the second horizontal grooves 332, limiting ribs 334 protruding out of the bottoms of the second horizontal grooves 33 are arranged at middle positions of the second horizontal grooves 332, and under the effect of an external force, the second connectors 71 surmount the limiting ribs 334 to be clamped in the limiting slots 333 of the second horizontal grooves 332, so that the heat-conduction cup 70 is fixedly connected with the receiving chamber 301. This connecting structure is more stable. When the heat-conduction cup 70 needs to be disassembled, the second connectors 71 will not be disengaged from the L-shaped clamping grooves 33 unless a large force is applied to make the second connectors 71 surmount the limiting ribs 334, so that the situation where the second connectors 71 are disengaged from the L-shaped clamping grooves 33 due to accidental touch or shaking, and consequentially, the heat-conduction cup 70 and the receiving chamber 301 are unstably connected, is effectively prevented.

Furthermore, in this embodiment, an open end face of the heat-conduction cup 70 is provided with two curved handle parts 72 which are symmetrically arranged and can be held by users by hand.

Furthermore, in this embodiment, the heat-conduction cup 70 is made from copper foil which is good in heat conductivity and stable in texture and structure. Definitely, in other embodiments, the copper foil can be replaced with other metal materials.

Furthermore, the atomization piece 40 is a ceramic atomization piece which is good in heat conductivity, stable in structure and long in service life. Definitely, in other embodiments, the atomization piece 40 may be a common atomization piece such as a glass glaze atomization piece or a stainless steel atomization piece which can also be used for heating and atomizing liquid.

Furthermore, the atomization piece 40 and the receiving chamber 301 are connected through an integrated filling formation process in the prior art, thereby being good in connection airtightness and easy and fast to manufacture. Definitely, in other embodiments, the atomization piece 40 and the receiving chamber 301 can also be subsequently assembled with a sealant or through other sealing methods.

Furthermore, in this embodiment, the shell comprises a base 10, an annular connecting base 20 and an atomizer base 30. The base 10 is provided with a receiving cavity 101, and an opening 102 penetrating through the receiving cavity 101 is formed in an upper portion of the base 10. The receiving chamber 301 is formed in the atomizer base 30 which is fixedly connected to the opening 102 of the base 10 through the annular connecting base 20. The electronic system is arranged in the receiving cavity 101 of the base 10.

Particularly, the atomizer base 30 is of a cylindrical structure, and a step part 31 extending outwards is formed on the outer wall of the bottom end of the atomizer base 30. The section of the annular connecting base 20 is in an L shape, and as shown in FIG. 3, the inner wall and the bottom surface of the annular connecting part 20 are fixedly attached to the step part 31 of the atomizer base 30. A connecting part 111 extending downwards is arranged at the opening 102 of the base 10, and the outer wall of the annular connecting base 20 is fixedly attached to the connecting part 111 at the opening 101 of the base 10, so that the base 10, the annular connecting base 20 and the atomizer base 30 are fixedly connected. When this connecting structure is used for assembling the base 10, the annular connecting base 20 and the atomizer base 30, the annular connecting base 20 abuts against the end of the connecting part 111 so as to be positioned, and after the annular connecting base 20 is positioned, the outer wall of the annular connecting base 20 is attached to the connecting part 111; then the atomizer base 30 is assembled by attaching the step part 31 of the atomizer base 30 to the inner wall and the bottom surface of the annular connecting base 20; and finally, the connecting structure is fixed through a conventional process such as bonding or welding process. Or, the inner wall and the bottom surface of the annular connecting base 20 are attached to the step part 31 of the atomizer base 30 first, realize positioned connection of the annular connecting base 20 and the atomizer base 30, then the annular connecting base 20 abuts against the end of the connecting part 111 to realize positioned connection of the base 10, the annular connecting base 20 and the atomizer base 30, and finally, the connecting structure is fixed through a conventional process such as bonding or welding. The connecting structure of the base 10, the annular connecting base 20 and the atomizer 30 is simple and reliable. Definitely, in other embodiments, the base 10, the annular connecting base 20, and the atomizer base 30 of the shell can also be connected through other connecting structures.

More particularly, the atomizer base 30 and the base 10 are made from plastic, thereby being light and portable and having a certain hardness. The annular connecting base 20 is an annular seal ring made from rubber, and the annular seal ring fulfills a transitional connection effect and a good sealing effect, so that the connecting structure of the atomizer base 30 and the base 10 has better airtightness. Or, in other embodiments, the atomizer base 30 and the base 10 can also be made from metals, carbon fibers, glass or the like, the annular connecting base 20 can be made from plastic, metals, carbon fibers or glass which has a certain hardness, and in this case, the annular connecting base 20 is mainly used for transitional connection and is connected with the atomizer base 30 and the base 10 with a sealant or through other sealing methods. Or, in other embodiments, the annular connecting base 20 is not adopted, and the base 10 and the atomizer base 30 are directly attached in a sealing manner with a sealant or are fixedly connected in other ways. This structure is the same as the structure in embodiment 1.

More particularly, the base 10 is composed of a lower cover 12 and an upper cover 11, the receiving cavity 101 is defined by the lower cover 12 and the upper cover 11 jointly, and the opening 102 is formed in the upper cover 11. When assembled, the upper cover 11 of the base 10, the atomizer base 30 and the annular connecting base 20 are assembled in the way mentioned above first, and then the upper cover 11 and the lower cover 12 are combined to complete assembly. Furthermore, the lower cover 12 is connected with the upper cover 11 and is covered with the upper cover 11, corresponding bolt holes are formed in the lower cover 12 and the upper cover 11, and the lower cover 12 and the upper cover 11 are connected through bolts. The connecting structure is simple, and operation is easy and convenient. Definitely, in other embodiments, the lower cover 12 and the upper cover 11 can also be fixedly connected through clamping, bonding or the like. The structure in this embodiment is the same as that in embodiment 1.

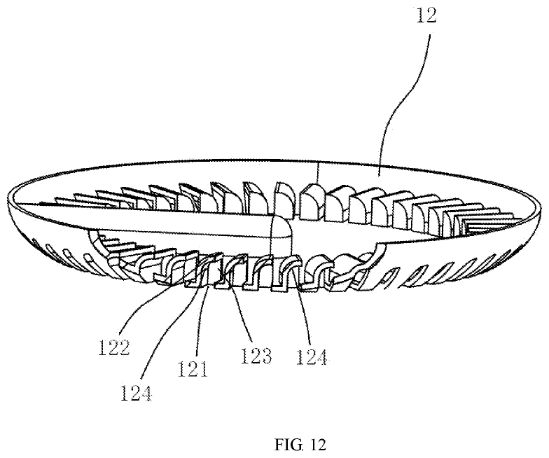

Furthermore, as shown in FIG. 12, the lower cover 12 is provided with heat dissipation slots 121 which are annularly arrayed, barrier parts 122 are integrally formed at positions, directly facing the heat dissipation slots 121, of the lower cover 12 of the base 10, and each barrier part 122 is provided with a lateral opening 123. The barrier parts 122 prevent the heat dissipation slots 121 from directly facing the receiving cavity 101, so that a good dust prevention effect is realized. When heat is dissipated, an external air flow enters the heat dissipation slots 121 and then flows into the receiving cavity 101 via the lateral openings 123, and an internal air flow enters the lateral openings 123 of the barrier parts 122 and then flows out via the heat dissipation slots 121. Meanwhile, the annularly-arrayed heat dissipation slots 121 can also fulfill uniform heat dissipation.

More particularly, each barrier part is provided with an arc-shaped inner wall 124 which is located between the corresponding heat dissipation slot 121 and the lateral opening 123 of the barrier part 122. When heat is dissipated, an external air flow enters the heat dissipation slots 121 and is then guided by the arc-shaped inner walls 124 to flow into the receiving cavity 101 via the lateral openings 123 of the barrier parts 122, and an internal air flow enters the lateral openings 123 of the barrier parts 122 and is then guided by the arc-shaped inner walls 124 to flow out via the heat dissipation slots 121. Through the configuration of the arc-shaped inner walls, the air flow can circulate more smoothly. This structure is the same as the structure in embodiment 1.

Furthermore, in this embodiment, clamping grooves 31 are concavely formed in the outer wall of the atomizer base 30 and are used for clamping a hood or other connecting structures. Particularly, each clamping groove 32 is in an L shape and comprises a first vertical groove 321 penetrating through the upper end face of the atomizer base 30 and a first horizontal groove 322 connected with the first vertical groove 321. First connectors (not shown) matched with the L-shaped clamping grooves 32 are arranged on the hood or other connecting structures. The first connectors are inserted via openings of the first vertical grooves 321 to reach the bottom and are then rotated to be clamped in the first horizontal grooves 322, so that clamping and fastening are realized. This connecting structure is simple and can fulfill connection rapidly. The structure in this embodiment is the same as the structure in embodiment 1.

Furthermore, in this embodiment, a switch button 60 is arranged on the shell and is connected with the electronic system so as to control the operation of the atomizer.

Particularly, the electronic system and the heating film of the atomizer are well known in the prior art and are common structures of existing atomizers (such as electronic cigarettes and humidifiers), the structure of controlling the operation of the atomizer by the switch button connected with the electronic system is also well known in the prior art and has already been mastered and actually applied by those skilled in this field, and thus, specific constituent parts and connection relations of these structures will not be detailed anymore.

### Embodiment 3

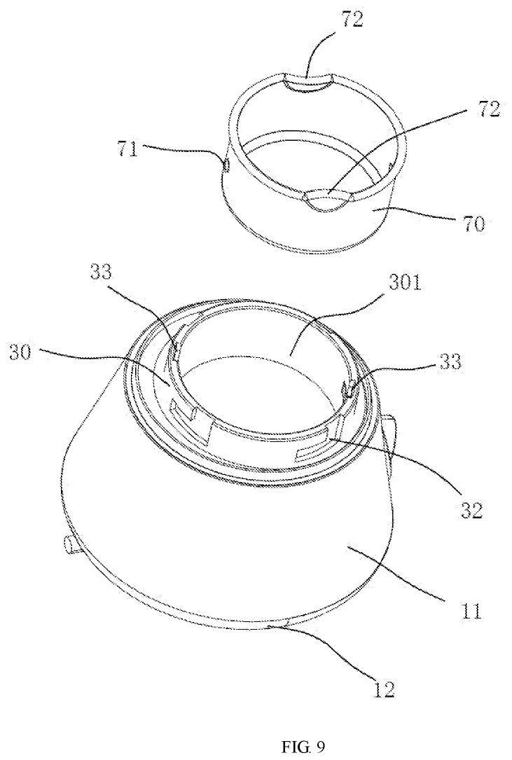

This embodiment provides an atomizer. The structure of the atomizer in this embodiment is basically the same as the structure in embodiment 2 and differs from the structure in embodiment 2 in that a respirator 80 is additionally arranged based on the structure in embodiment 2 as shown in FIG. 9, so that aerosols formed by atomization can be inhaled by users, and applications of the atomizer in the field of medical instruments are realized. The respirator 80 and the atomizer base 30 are detachably and fixedly connected particularly through the clamping grooves 32 and the first connectors in embodiment 1, namely, the first connectors are arranged on the inner wall of the respirator 80 and are clamped in and matched with the clamping grooves 32 formed in the atomizer base 30. Definitely, the respirator 80 and the atomizer base 30 can also be connected in other ways such as buckling and screwing.

On the basis of the structure in embodiment 2, the respirator is additionally arranged to obtain the structure in this embodiment to fulfill the specific application of the atomizer. Definitely, in other embodiments, the respirator can also be additionally arranged on the basis of the structure in embodiment 1. Or, the respirator can be replaced with other specific structures to full corresponding applications. For instance, a nozzle can be additionally arranged for mist injection, and in this case, the atomizer can be used for optimizing and improving the environment by humidification or aroma.

Although the invention is specifically illustrated and introduced above in combination with preferred embodiments, those skilled in this field would appreciate that various variations of the invention can be made in forms and in details without deviating from the spirit and scope defined by the claims, and all these variations should also fall within the protection scope of the invention.

What is claimed is:

1. An atomizer, comprising a shell, an electronic system and a heating body, wherein the heating body comprises an atomization piece of a flat structure and a heating film attached to a bottom surface of the atomization piece, the shell is provided with a receiving chamber, the atomization piece is matched with a bottom of the receiving chamber in shape and is laid at the bottom of the receiving chamber in a sealing manner, the bottom of the receiving chamber is hollowed out to form a through hole, the heating film is exposed out of the through hole, and the electronic system is arranged in the shell and is electrically connected with the heating film.

2. The atomizer according to claim 1, wherein a metal heat-conduction layer is laid on an inner wall of the receiving chamber and an outer edge of the atomization piece in a sealing manner.

3. The atomizer according to claim 2, wherein the metal heat-conduction layer is a copper foil layer.

4. The atomizer according to claim 1, wherein a heat-conduction cup is arranged in the receiving chamber of the shell, and the atomization piece is attached to a bottom surface of the heat-conduction cup.

5. The atomizer according to claim 4, wherein an outer wall of the heat-conduction cup is matched with an inner wall of the receiving chamber.

6. The atomizer according to claim 4, wherein the heat-conduction cup is detachably installed in the receiving chamber of the shell.

7. The atomizer according to claim 6, wherein the heat-conduction cup is provided with first installation structures, the receiving chamber of the shell is provided with second installation structures, and the heat-conduction cup is installed in the receiving chamber through the cooperation between the first installation structures of the heat-conduction cup and the second installation structures of the receiving chamber.

8. The atomizer according to claim 7, wherein the first installation structures are second connectors arranged on the outer wall of the heat-conduction cup, and the second installation structures are L-shaped clamping grooves formed in the inner wall of the receiving chamber; each said L-shaped clamping groove comprises a second vertical groove penetrating through an upper end face of an atomizer base and a second horizontal groove connected with the second vertical groove; and the second connectors are inserted via the second vertical grooves to be clamped into the second horizontal grooves.

9. The atomizer according to claim 8, wherein limiting slots matched with the second connectors are formed in tail ends of the second horizontal grooves, limiting ribs protruding out of bottoms of the second horizontal grooves are arranged at middle positions of the second horizontal grooves, and under the effect of an external force, the second connectors surmount the limiting ribs to be clamped in the limiting slots of the second horizontal grooves.

10. The atomizer according to claim 8, wherein openings, penetrating through the upper end face of the atomizer base, of the second vertical grooves are open.

11. The atomizer according to claim 1, wherein the atomization piece and receiving chamber are connected through integrated filling formation

12. The atomizer according to claim 1, wherein the atomization piece is a ceramic atomization piece.

13. The atomizer according to claim 1, wherein the shell comprises a base, an annular connecting base and an atomizer base, the base is provided with a receiving cavity, an opening penetrating through the receiving cavity is formed in an upper portion of the base, the receiving chamber is formed in the atomizer base which is fixedly connected to the opening of the base through the annular connecting base, and the electronic system is arranged in the receiving cavity of the base.

14. The atomizer according to claim 13, wherein the base is provided with heat dissipation slots.

15. The atomizer according to claim 14, wherein the number of the heat dissipation slots is more than one, and the heat dissipation slots are annularly arrayed on the base.

16. The atomizer according to claim 14, wherein barrier parts are integrally formed at positions, directly facing the heat dissipation slots, of the base, and each said barrier part is provided with a lateral opening.

17. The atomizer according to claim 16, wherein each said barrier part is further provided with an arc-shaped inner wall located between the corresponding heat dissipation slot and the lateral opening of the barrier part.

18. The atomizer according to claim 14, wherein the base is composed of a lower cover and an upper cover, the receiving cavity is defined by the lower cover and the upper cover jointly, the opening is formed in the upper cover, and the heat dissipation slots are formed in the lower cover.

19. The atomizer according to claim 13, wherein clamping grooves are concavely formed in an outer wall of the atomizer base.

20. The atomizer according to claim 1, wherein a switch button is arranged on the shell and is connected with the electronic system.
